(19) United States
(12) Patent Application Publication
(10) Pub. No.: US 2023/0033276 A1
(43) Pub. Date: Feb. 2, 2023

(54) ACTIVE INGREDIENT-CONTAINING NANOEMULSIONS

(71) Applicant: NICOVENTURES TRADING LIMITED, London (GB)

(72) Inventors: Keyi Xu, Whitsett, NC (US); Thomas H. Poole, Winston-Salem, NC (US)

(21) Appl. No.: 17/870,228

(22) Filed: Jul. 21, 2022

Related U.S. Application Data
(60) Provisional application No. 63/224,750, filed on Jul. 22, 2021.

(51) Int. Cl. A61K 9/107; A61K 31/05; A61K 47/12; A61K 47/44

(52) U.S. Cl. CPC A61K 9/1075; A61K 31/05; A61K 47/12; A61K 47/44; B82Y 5/00

(57) ABSTRACT

The present disclosure provides nanoemulsions including one or more active ingredients (e.g., one or more cannabinoid and/or cannabimimetics), formulated for use as tinctures or shots. Such nanoemulsions, tinctures, and shots can exhibit high physical stability, e.g., exhibiting no observable phase separation over a period of 6 months or more or 12 months or more.

### CROSS REFERENCE TO RELATED APPLICATIONS

The present application claims priority to and the benefit of U.S. Provisional Patent Application No. 63/224,750, filed on Jul. 22, 2021, the disclosure of which is incorporated herein by reference in its entirety.

### FIELD OF THE DISCLOSURE

The present disclosure relates to emulsions comprising one or more active ingredients and to products including such emulsions.

### BACKGROUND

Tobacco and various active ingredients may be enjoyed in a so-called “smokeless” form. Particularly popular oral tobacco-containing products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Oral product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference. Oral products in similar formats and which are free of tobacco have also been proposed.

It would be desirable to provide products configured for oral use which may deliver active ingredients to the consumer in alternative forms.

### BRIEF SUMMARY

The present disclosure relates generally to emulsions comprising one or more active ingredients, as well as to products comprising such emulsions. For example, in some embodiments, such liquids are emulsions, e.g., nanoemulsions. The nanoemulsions can comprise one or more active ingredients in concentrated form (e.g., in the form of a “tincture”), and/or can be diluted to provide the one or more active ingredients in diluted form (e.g., in the form of a “shot”). Advantageously, the disclosed liquids exhibit high stability as evaluated, e.g., via physical observation.

The present disclosure includes, without limitation, the following embodiments:

Embodiment 1: A nanoemulsion, comprising: a cannabinoid and/or a cannabimimetic; a first oil; a second oil; and water, wherein the cannabinoid or cannabimimetic is present in an amount of about 1% by weight or greater.

Embodiment 2: The nanoemulsion of Embodiment 1, wherein the cannabinoid or cannabimimetic is present in an amount of about 2% by weight or greater.

Embodiment 3: The nanoemulsion of Embodiment 1, wherein the cannabinoid or cannabimimetic is present in an amount of about 1% to about 4% by weight.

Embodiment 4: The nanoemulsion of Embodiment 1, wherein the cannabinoid or cannabimimetic is present in an amount of about 2% to about 3% by weight.

Embodiment 5: The nanoemulsion of any of Embodiments 1-4, wherein the cannabinoid or cannabimimetic is selected from the group consisting of cannabigerols, cannabichromenes, cannabidiols, tetrahydrocannabinols, cannabinols, cannabinodiols, and combinations thereof.

Embodiment 6: The nanoemulsion of any of Embodiments 1-4, wherein the cannabinoid or cannabimimetic is selected from the group consisting of cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), tetrahydrocannabivarinic acid (THCV A), and combinations thereof.

Embodiment 7: The nanoemulsion of any of Embodiments 1-4, wherein the cannabinoid or cannabimimetic comprises cannabidiol (CBD).

Embodiment 8: The nanoemulsion of any of Embodiments 1-4, wherein the cannabinoid or cannabimimetic is selected from the group consisting of yangonin, alpha-amyrin or beta-amyrin (also classified as terpenes), cyanidin, curcumin (turmeric), catechin, quercetin, salvinorin A, N-acylethanolamines, N-alkylamide lipids, and combinations thereof.

Embodiment 9: The nanoemulsion of any of Embodiments 1-8, The nanoemulsion of any of claims 1-8, wherein first oil comprises sunflower oil.

Embodiment 10: The nanoemulsion of any of Embodiments 1-9, wherein the second oil comprises lecithin.

Embodiment 11: The nanoemulsion of any of Embodiments 1-10, wherein the second oil is canola lecithin.

Embodiment 12: The nanoemulsion of any of Embodiments 1-11, wherein the weight ratio of the second oil to the first oil is about 1.5 or greater.

Embodiment 13: The nanoemulsion of any of Embodiments 1-12, wherein the weight ratio of the second oil to the first oil is about 1.5 to about 3.

Embodiment 14: The nanoemulsion of any of Embodiments 1-13, further comprising a surfactant.

Embodiment 15: The nanoemulsion of Embodiment 14, wherein the surfactant is a polyoxyethylene stearate.

Embodiment 16: The nanoemulsion of any of Embodiments 1-15, further comprising one or more natural or artificial sweeteners.

Embodiment 17: The nanoemulsion of Embodiment 16, wherein the natural or artificial sweetener is selected from the group consisting of saccharin, acesulfame K, aspartame, sucralose, isomalt, lactose, mannitol, sorbitol, xylitol, sucrose, Stevia, and combinations thereof.

Embodiment 18: The nanoemulsion of Embodiment 17, wherein the natural or artificial sweetener comprises Stevia.

Embodiment 19: The nanoemulsion of any of Embodiments 1-18, further comprising a bitterness suppressant.

Embodiment 20: The nanoemulsion of Embodiment 19, wherein the bitterness suppressant is a glycyrrhizic acid or a salt thereof.

Embodiment 21: The nanoemulsion of any of Embodiments 1-20, further comprising an antioxidant.

Embodiment 22: The nanoemulsion of Embodiment 21, wherein the antioxidant is selected from the group consisting of wherein the antioxidant is selected from the group consisting of citric acid, Vitamin E, a tocopherol, epicatechol, epigallocatechol, epigallocatechol gallate, erythorbic acid, sodium erythorbate, ascorbyl palmitate, ascorbyl stearate, sodium ascorbate, ascorbic acid, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

Embodiment 23: The nanoemulsion of any of Embodiments 1-22, further comprising a humectant.

Embodiment 24: the nanoemulsion of Embodiment 23, wherein the humectant is a polyol.

Embodiment 25: The nanoemulsion of Embodiment 23, wherein the humectant comprises glycerin, propylene glycol, 1,3-propanediol, dipropylene glycol, sorbitol, xylitol, mannitol, or a combination thereof.

Embodiment 26: The nanoemulsion of any of Embodiments 1-25, wherein the nanoemulsion is substantially free of a mono-alcohol (e.g., including, but not limited to, ethanol).

Embodiment 27: The nanoemulsion of any of Embodiments 1-26, wherein the nanoemulsion comprises a dispersed phase within the water, wherein the dispersed phase has an average droplet size of about 100 nm or less.

Embodiment 28: The nanoemulsion of any of Embodiments 1-27, wherein the nanoemulsion exhibits physical stability for 6 months or more.

Embodiment 29: The nanoemulsion of any of Embodiments 1-27, wherein the nanoemulsion exhibits physical stability for 12 months or more.

Embodiment 30: An oral product in the form of a tincture, consisting essentially of the nanoemulsion of any of Embodiments 1-29.

Embodiment 31: An oral product in the form of a shot, comprising the nanoemulsion of any of Embodiments 1-29, diluted in water.

Embodiment 32: The oral product of Embodiment 31, further comprising at least one buffering agent, at least one preservative, at least one antioxidant, or any combination thereof (e.g., including at least one buffering agent, at least one preservative, and at least one antioxidant).

Embodiment 33: The oral product of any of Embodiments 31-32, wherein the shot exhibits physical stability for 6 months or more.

Embodiment 34: The oral product of any of Embodiments 31-33, wherein the shot exhibits physical stability for 12 months or more.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present disclosure will become apparent from the following.

### BRIEF DESCRIPTION OF THE DRAWINGS

Having thus described aspects of the disclosure in the foregoing general terms, reference will now be made to the accompanying drawings, which are not necessarily drawn to scale. The drawings are examples only, and should not be construed as limiting the disclosure.

FIG. 1 is a dynamic light scattering plot for an example of a nanoemulsion provided according to one embodiment of the disclosure;

FIG. 2 is a dynamic light scattering plot for an example of an aged nanoemulsion provided according to one embodiment of the disclosure; and

FIG. 3 is a dynamic light scattering plot for an example of a diluted nanoemulsion (in the form of a drinkable shot) provided according to one embodiment of the disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to percent is intended to mean percent by weight unless otherwise indicated.

As described hereinafter, example embodiments of the present disclosure relate to liquids comprising one or more active ingredients. The liquids can be used directly (e.g., in the form of tinctures for delivery of the active agent) or can be further processed (e.g., by diluting to form a shot for delivery of the active agent or by further processing to incorporate the liquid within another type of product (e.g., within a food, beverage, liquid-filled oral capsule, or the like).

In some embodiments, such liquids are suitable/configured for oral use/consumption, e.g., to be inserted directly into the subject's mouth or can be further formulated or processed for inclusion within a product, which product is inserted into the subject's mouth. In certain embodiments, the liquid (or product comprising such liquid) is adapted to deliver components to a subject through mucous membranes in the subject's mouth and/or through the subject's digestive system and, in some instances, said component is an active ingredient that can be absorbed through the mucous membranes in the mouth and/or the digestive system when the liquid (or product comprising the liquid) is used.

The liquids provided herein are generally in the form of emulsions and, in particular, in the form of nanoemulsions. The terms “emulsion” and “nanoemulsion” have their usual meaning in the chemical and pharmaceutical arts. For example, an emulsion can be described as a dispersion of droplets of one liquid (the dispersed phase) suspended in a second liquid (the continuous phase), wherein the two liquids are normally immiscible with one another. The droplets are generally spherical. Emulsions can generally be oil-in-water-type emulsions (where the dispersed phase comprises an oil and the continuous phase comprises water), and water-in-oil-type emulsions (where the dispersed phase comprises water and the continuous phase comprises an oil). The presently disclosed emulsions are typically oil-in-water-type emulsions.

A nanoemulsion is an emulsion with droplet sizes within the range of about 20 nm to about 500 nm (often about 100 nm to about 500 nm, e.g., about 100 nm to about 200 nm). In various embodiments, nanoemulsions provided herein exhibit low droplet sizes, e.g., with Z-average droplet sizes below about 100 nm (e.g., about 50 nm to about 100 nm). Such low droplet sizes are advantageous, e.g., in delivery of active agents (as provided herein below). For example, the disclosed nanoemulsions allow active ingredients (and in particular, highly lipophilic active ingredients) to be more readily absorbed due to the small droplet sizes of these nanoemulsions. Advantageously in some embodiments, the droplet sizes (diameters) of the disclosed nanoemulsions are substantially uniform, i.e., with low polydispersity.

The nanoemulsions of the present disclosure comprise multiple components. The nanoemulsions generally comprise an active ingredient, a surfactant, a lipid component, and water. Typically, the active ingredient is contained within the dispersed phase. The droplets (also referred to herein as “micelles” or “particles”) of the dispersed phase can maintain solubility of the active ingredient during ingestion, e.g., in the aqueous stomach environment and can protect the active ingredient from enzymes in the stomach and intestines. Eventually, these droplets are transported to the intestinal lining where they are absorbed; as noted above, the small droplet size renders the disclosed nanoemulsions advantageous in providing ready absorption of the active ingredient. Each of these components will be described more fully herein below with non-limiting examples, and additional components that can optionally be incorporated within the disclosed nanoemulsions will be disclosed as well.

### Active Ingredient

One component of the nanoemulsions and related products provided herein is an active agent (also referred to herein as an “active ingredient”). The present disclosure is particularly advantageous in the context of active ingredients that are hydrophobic and/or lipophilic. Such active ingredients are not readily provided in aqueous solution and thus, delivery of such active ingredients can be challenging. The compositions and methods outlined herein allow for the delivery of such hydrophobic and/or lipophilic active ingredients via a liquid (i.e., nanoemulsion) form. Within the general class of active ingredients, one will recognize that certain examples are hydrophilic and certain examples are hydrophobic; similarly, certain examples are lipophilic and certain examples are lipophobic. The principles provided herein are particularly applicable in the context of those examples that are substantially hydrophobic and/or those examples that are substantially lipophilic.

The active ingredient can be any known agent adapted for therapeutic, prophylactic, or diagnostic use. These can include, for example, synthetic organic compounds, proteins and peptides, polysaccharides and other sugars, lipids, inorganic compounds, and nucleic acid sequences, having therapeutic, prophylactic, or diagnostic activity. Example active ingredients would include any ingredient known to impact one or more biological functions within the body, such as ingredients that furnish pharmacological activity or other direct effect in the diagnosis, cure, mitigation, treatment, or prevention of disease, or which affect the structure or any function of the body of humans or other animals (e.g., provide a stimulating action on the central nervous system, have an energizing effect, an antipyretic or analgesic action, or an otherwise useful effect on the body). Active ingredients include, but are not limited to cannabinoids and cannabimimetics.

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in Cannabis; and synthetic cannabinoids, manufactured artificially. Cannabinoids found in Cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in Cannabis, and cannabidiol (CBD) another major constituent of the plant, but which is devoid of psychoactivity. All of the above compounds can be used in the form of an isolate from plant material or synthetically derived.

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid comprises at least tetrahydrocannabinol (THC). In some embodiments, the cannabinoid is tetrahydrocannabinol (THC). In some embodiments, the cannabinoid comprises at least cannabidiol (CBD). In some embodiments, the cannabinoid is cannabidiol (CBD). In some embodiments, the CBD is synthetic CBD.

Alternatively (or in addition), the active ingredient can be a cannabimimetic, which is a class of compounds derived from plants other than Cannabis that have biological effects on the endocannabinoid system similar to cannabinoids. Examples include yangonin, alpha-amyrin or beta-amyrin (also classified as terpenes), cyanidin, curcumin (turmeric), catechin, quercetin, salvinorin A, N-acylethanolamines, and N-alkylamide lipids. Such compounds can be used in the same amounts and ratios noted herein for cannabinoids.

The choice of cannabinoid or cannabimimetic and the particular percentages thereof which may be present within the disclosed oral product will vary depending upon the desired flavor, texture, and other characteristics of the oral product.

In some embodiments, the cannabidiol is synthetic cannabidiol. In some embodiments, the cannabinoid is used in the form of an isolate. In some embodiments, the cannabinoid (e.g., cannabidiol, CBD) is added to the nanoemulsion in the form of an isolate. An isolate is an extract from a plant, such as Cannabis, where the active material of interest (in this case the cannabinoid, such as CBD) is present in a high degree of purity, for example greater than 95%, greater than 96%, greater than 97%, greater than 98%, or around 99% purity. In some embodiments, the cannabinoid is an isolate of CBD in a high degree of purity, and the amount of any other cannabinoid in the oral product is no greater than about 1% by weight of the oral product, such as no greater than about 0.5% by weight of the oral product, such as no greater than about 0.1% by weight of the oral product, such as no greater than about 0.01% by weight of the oral product.

A cannabinoid (e.g., CBD) or cannabimimetic is typically in a concentration within the disclosed nanoemulsions in an amount of at least about 0.5% by weight, e.g., at least about 1% by weight, at least about 1.1% by weight, at least about 1.2% by weight, at least about 1.3% by weight, at least about 1.4% by weight, at least about 1.5% by weight, at least about 1.6% by weight, at least about 1.7% by weight, at least about 1.8% by weight, at least about 1.9% by weight, at least about 2% by weight, at least about 2.1% by weight, at least about 2.2% by weight, at least about 2.3% by weight, at least about 2.4% by weight, or at least about 2.5% by weight, with an upper limit of, e.g., about 5% by weight, about 4% by weight, about 3% by weight, about 2.9% by weight, about 2.8% by weight, about 2.7% by weight, or about 2.6% by weight. In some embodiments, the composition as disclosed herein comprises a cannabinoid, e.g., CBD, in an amount from about 1% by weight to about 3% by weight, or from about 2% to about 3% by weight, based on the total weight of the nanoemulsion.

In some embodiments, the nanoemulsion may comprise a further active ingredient in combination (or in place of) the cannabinoid or cannabimimetic. In some embodiments, two or more active ingredients can be incorporated within the same nanoemulsion. For example, in some embodiments, the nanoemulsion may include one or more active ingredients in addition to a cannabinoid or cannabimimetic. Such active ingredients can be, e.g., APIs (active pharmaceutical ingredients), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C). Each of these categories is further described herein below. The particular choice of active ingredients may vary depending upon the desired characteristics of the particular nanoemulsion.

In some embodiments, the nanoemulsion comprises as an active ingredient (e.g., in addition to a cannabinoid and/or cannabamimetic) a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the nanoemulsions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, or about 5% by weight, based on the total weight of the composition.

Botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, Cordyceps, curcumin, damiana, Dorstenia arifolia, Dorstenia odorata, essential oils, Eucalyptus, fennel, Galphimia glauca, ginger, Ginkgo biloba, ginseng (e.g., Panax ginseng), green tea, Griffonia simplicifolia, guarana, Cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the nanoemulsions comprise lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the nanoemulsions comprise lemon balm extract. In some embodiments, the lemon balm extract is present in an amount of from about 0.1 to about 4% by weight, based on the total weight of the composition.

In some embodiments, the nanoemulsions comprise ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the nanoemulsion.

In some embodiments, the nanoemulsions comprise one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure®, available from Balchem Corp., 52 Sunrise Park Road, New Hampton, N.Y., 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the nanoemulsion.

In some embodiments, the nanoemulsions comprise, as an active ingredient, an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient comprises a combination of theanine and tryptophan. In some embodiments, the active ingredient comprises a combination of theanine and one or more B vitamins. In some embodiments, the nanoemulsions comprise a combination of caffeine, theanine, and optionally, ginseng. In some embodiments, the active ingredient comprises taurine. In some embodiments, the active ingredient is a combination of caffeine and taurine.

Without being bound by any theory of operation, it is believed that certain amino acids, such as theanine, tryptophan, GABA, or taurine, can have beneficial impact on mood, anxiety level, focus, or cognitive performance, particularly when combined with other active ingredients, such as caffeine or certain botanicals.

When present, an amino acid or combination of amino acids (e.g., theanine, taurine, GABA, tryptophan, and combinations thereof) is typically at a concentration of from about 0.01% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the nanoemulsion. In one embodiment, the at least one active ingredient comprises tryptophan in an amount by weight from about 0.03% to about 1%, or from about 0.05% to about 0.5%.

In some embodiments, the nanoemulsion comprises, as an active ingredient, a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine. In some embodiments, the active ingredient comprises one or more of vitamin B6 and B12. In some embodiments, the active ingredient comprises theanine and one or more of vitamin B6 and B12. When present, a vitamin or combination of vitamins (e.g., vitamin B6, vitamin B12, vitamin E, vitamin C, or a combination thereof) is typically at a concentration of from about 0.0001% to about 6% by weight, such as, e.g., from about 0.0001, about 0.001, about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5%, about 0.6%, about 0.7%, about 0.8%, about 0.9%, about 1%, about 2%, about 3%, about 4%, about 5%, or about 6% by weight, based on the total weight of the composition.

In some embodiments, the nanoemulsion comprises vitamin B6 in an amount from about 0.008% to about 0.06% by weight, or from about 0.01% to about 0.04% by weight. In some embodiments, the active ingredient comprises vitamin B12 in an amount from about 0.0001% to about 0.007% by weight, or from about 0.0005% to about 0.001% by weight. In some embodiments, the active ingredient comprises a combination of vitamin B6 and vitamin B12 in a total amount by weight from about 0.008% to about 0.07%. In some embodiments, the nanoemulsion comprises vitamin A. In some embodiments, the vitamin A is encapsulated.

In some embodiments, the nanoemulsion comprises, as an active ingredient, a mineral. As used herein, the term “mineral” refers to an inorganic molecule (or related set of molecules) that is an essential micronutrient needed for the proper functioning of various systems in a mammal. Non-limiting examples of minerals include iron, zinc, copper, selenium, chromium, cobalt, manganese, calcium, phosphorus, sulfur, magnesium, and the like. In some embodiments, the active ingredient comprises iron. Suitable sources of iron include, but are not limited to, ferrous salts such as ferrous sulfate and ferrous gluconate. In some embodiments, the iron is encapsulated.

In certain embodiments, the nanoemulsion comprises, as an active ingredient, a nicotine component. By “nicotine component” is meant any suitable form of natural or synthetic nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

Typically, the nicotine component (calculated as the free base) when present, is in a concentration of at least about 0.001% by weight of the nanoemulsion, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the nanoemulsion. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition. In some embodiments, the nanoemulsions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the disclosed nanoemulsions can comprise, as an active ingredient, one or more terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active ingredient comprises more than one terpene. For example, the active ingredient may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

In some embodiments, the nanoemulsion comprises, as an active ingredient, an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the nanoemulsion comprises citicoline. In some embodiments, the nanoemulsion comprises a combination of citicoline, caffeine, theanine, and ginseng. In some embodiments, the active ingredient comprises sunflower lecithin. In some embodiments, the nanoemulsion comprises a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the nanoemulsion.

In some embodiments, the nanoemulsion is substantially free of any API. By “substantially free of any API” means that the nanoemulsion does not contain, and specifically excludes, the presence of any API as defined herein, such as any Food and Drug Administration (FDA) approved therapeutic agent intended to treat any medical condition. For example, certain embodiments can be characterized as having less than 0.001% by weight of an API, or less than 0.0001%, or even 0% by weight of an API.

### Oils

The nanoemulsions described herein comprise two or more oils. Advantageously, the active ingredient is at least partially soluble in at least one of the two or more oils. Advantageously, both the first oil and the second oil in various embodiments are food grade oils, including, e.g., fractionated oils. In certain embodiments, the first and/or second oils are selected so as to minimize allergic reactions.

A first oil can be selected from various types of oils including, but not limited to, vegetable oils (e.g., acai oil, almond oil, amaranth oil, apricot oil, apple seed oil, argan oil, avocado oil, babassu oil, beech nut oil, ben oil, bitter gourd oil, black seed oil, blackcurrant seed oil, borage seed oil, borneo tallow nut oil, bottle gourd oil, brazil nut oil, buffalo gourd oil, butternut squash seed oil, cape chestnut oil, canola oil, carob cashew oil, castor oil, cocoa butter, cocklebur oil, coconut oil, corn oil, cothune oil, coriander seed oil, cottonseed oil, date seed oil, dika oil, egus seed oil, evening primrose oil, false flax oil, flaxseed oil, grape seed oil, grapefruit seed oil, hazelnut oil, hemp oil, kapok seed oil, kenaf seed oil, lallemantia oil, lemon oil, linseed oil, macadamia oil, mafura oil, manila oil, meadowfoam seed oil, mongongo nut oil, mustard oil, niger seed oil, nutmeg butter, okra seed oil, olive oil, orange oil, palm oil, Papaya seed oil, peanut oil, pecan oil, Perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, ramtil oil, rapeseed oil, rice bran oil, royle oil, sacha inchi oil, safflower oil, sapote oil, seje oil, sesame oil, shea butter, soybean oil, sunflower oil, taramira oil, tea seed oil, thistle oil, tigernut oil, tobacco seed oil, tomato seed oil, walnut oil, watermelon seed oil, wheat germ oil, and combinations thereof), animal oils (e.g., cattle fat, buffalo fat, sheep fat, goat fat, pig fat, lard, camel fat, tallow, liquid margarine, fish oil, fish liver oil, whale oil, seal oil, and combinations thereof), and mineral oils. The oil may, in some embodiments, be an omega 3 oil. In certain embodiments, a first oil is selected that provides for good dissolution of the active ingredient. One example of a suitable first oil is sunflower oil.

A second oil can be similarly selected. In certain embodiments, the second oil comprises lecithin. Lecithin is generally a mixture of phospholipids in oil (e.g., phosphatidylcholine, phosphatidylethanolamine, and phosphatidylinositol), which can be obtained, e.g., by degumming oils. The composition of lecithin can vary depending upon the origin of the lecithin. Although lecithin is described herein as an example of a “second oil,” it is noted that lecithin is not, in fact, necessarily derived from the corresponding oil. For example, canola lecithin is derived from canola cultivars of rapeseed. Further, in some embodiments, the second oil may function, at least in part, as a surfactant. Examples of lecithins include, but are not limited to, sunflower lecithin, soybean lecithin, and canola lecithin.

Lecithins are available in natural/crude and refined grades (which are typically heat processed, thereby removing, e.g., proteins from the lecithin). In certain embodiments, natural/crude grades are desirable over refined grades. In some embodiments, use of a crude, unrefined lecithin (e.g., canola lecithin) surprisingly leads to a nanoemulsion with greater physical stability than that of a nanoemulsion prepared using a refined lecithin. Further, use of canola lecithin (as opposed to, e.g., sunflower lecithin, soy lecithin, and/or purified phosphatidylcholine) as the second oil surprisingly was found to lead to a nanoemulsion with greater physical stability.

The first and second oils of the disclosed nanoemulsions can be provided in varying amounts overall and in varying ratios with respect to one another. In some embodiments, the first and second oils together comprise about 10% or more by weight, about 15% or more by weight, about 16% or more by weight, about 17% or more by weight, about 18% or more by weight, about 19% or more by weight, or about 20% or more by weight, e.g., including about 10% to about 25% by weight or about 15% to about 22% by weight. In some embodiments, the first and second oils are in roughly equal proportions to one another by weight. In other embodiments, the nanoemulsion comprises a higher weight percentage of the first oil or the second oil. For example, in some embodiments, the lecithin is provided in a greater weight percentage than the first oil (e.g., at least about 1.1 times the amount of the first oil, at least about 1.3 times the amount of the first oil, or at least about 1.5 times the amount of the first oil). Non-limiting ranges of weight ratios of the lecithin to the first oil include ratios of about 1.1:1 to about 2:1, e.g., about 1.2:1 to about 2:1, about 1.3:1 to about 2:1, about 1.4:1 to about 2:1, or about 1.5:1 to about 2:1.

The nanoemulsions are not limited to comprising two oils; in some embodiments, the nanoemulsions can comprise three or more or four or more oils. Such additional oils can be selected from the listing provided above.

### Surfactant

A surfactant is generally included in the disclosed nanoemulsions and can function to help in stabilizing the nanoemulsion. Surfactants can be included in the continuous phase, the dispersed phase, or both phases of a nanoemulsion. Surfactant molecules typically comprise both hydrophilic and hydrophobic regions, and can thus adsorb at oil-water interfaces, reducing interfacial tension and forming a protective layer around droplets of the dispersed phase within a nanoemulsion. Surfactants can be ionic or nonionic. In certain embodiments, the disclosed nanoemulsions comprise one or more anionic surfactants. Examples of anionic surfactants include, e.g., sulfates, sulfonates, and carboxylates (with counter ions such as ammonium, sodium, or potassium cations). Certain specific types of anionic surfactants are lauryl/laureth sulfates, and alkyl sulfates, alkyl ether sulfates, alpha-olefin sulfonates. Surfactants can be further classified as hydrophilic or hydrophobic.

Examples of surfactants that can be used in the disclosed nanoemulsions include, but are not limited to long-chain triglycerides, such as C16-C18 triglycerides, linoleic acid, glyceryl monooleate, sodium lauryl sulfate (sodium dodecyl sulfate, SLS, or SDS), docusate sodium, polyoxyethylene sorbitan fatty acid ester surfactants (including, e.g., mono- and tri-lauryl, palmityl, stearyl and oleyl esters), such as those known as polysorbates and commercially available under the tradename TWEEN® (e.g., TWEEN®20, TWEEN®40, TWEEN®65, TWEEN®80, and TWEEN®85); polyoxyethylene fatty acid esters, e.g., polyoxyethylene stearic acid esters such as those commercially available under the trade name MYRJ™ (e.g., MYRJ™ 52); polyoxyethylene ethers, such as those available under the trade name BRIJ® (e.g., BRIJ® 23, BRIJ® 30, BRIJ® 35, BRIJ® 52, BRIJ® 56, BRIJ® 58, BRIJ® 72, and BRIJ® 78); polyoxyethylene castor oil derivatives, e.g., those commercially available as CREMOPHOR® surfactants (e.g., CREMOPHOR® EL, CREMOPHOR® ELP, and CREMOPHOR® RH40); sorbitan fatty acid esters, such as those commercially available under the tradename SPAN® (e.g., SPAN®20, SPAN®40, SPAN®60, SPAN®65, SPAN®80, and SPAN®85); PEG glyceryl fatty acid esters such as PEG-8 glyceryl caprylate/caprate (commercially known as LABRASOL®); polyoxyethylene-polyoxypropylene co-polymers, e.g., those commercially available as PLURONIC® or POLOXAMER®; diethyleneglycol-monoethylether (DGME), commercially known as TRANSCUTOL®; polyoxyethylene 15 hydroxy stearate (Macrogol 15 hydroxy stearate, Solutol HS15®); polyoxyethylene nonylphenol ether (NONOXYNOL®); PEG-4 glyceryl caprylate/caprate (Labrafac Hydro WL 1219); PEG-32 glyceryl laurate (Gelucire 44/14); PEG-6 glyceryl mono oleate (Labrafil® M 1944 CS); PEG-6 glyceryl linoleate (Labrafil® M 2125 CS); monoglycerides and acetylated monoglycerides, e.g., glycerol monodicocoate (IMWITOR® 928) and glycerol monocaprylate (IMWITOR® 308); mono- and di-acetylated monoglycerides; α-tocopherol; α-tocopheryl polyethylene glycol succinate (vitamin E TPGS); α-tocopherol palmitate and α-tocopherol acetate; propylene glycol mono- and di-fatty acid esters, such as propylene glycol laurate; propylene glycol caprylate/caprate; glycerol triacetate; sugar esters, lecithins, and combinations of any two or more thereof. In some embodiments, a combination of two or more surfactants is included in the disclosed nanoemulsions.

The amount of surfactant in the disclosed nanoemulsions can vary. In some embodiments, the amount of surfactant is about 2% or greater by weight, about 3% or greater by weight, about 4% or greater by weight, about 5% or greater by weight, about 6% or greater by weight, about 7% or greater by weight, about 8% or greater by weight, or about 9% or greater by weight. In some embodiments, the amount of surfactant is no more than about 25% by weight, no more than about 20% by weight, no more than about 15% by weight, no more than about 14% by weight, no more than about 13% by weight, no more than about 12% by weight, no more than about 11% by weight, or no more than about 10% by weight. Certain, non-limiting ranges include, e.g., about 2% by weight to about 25% by weight, about 5% to about 20% by weight, or about 5% to about 15% by weight. In some embodiments, the amount of surfactant is varied to obtain a suitable nanoemulsion exhibiting the properties outlined herein.

### Water

As described herein, the disclosed nanoemulsions comprise water, and generally comprise water in the continuous phase thereof. Water may be present as, for example, purified or ultrapure water, saline, buffered saline, or a buffered aqueous phase. In some embodiments, water is the predominant component by weight (i.e., water is higher in a greater amount than any of the other individual components). However, the total content of water may be less than about 50% by weight of the nanoemulsion.

The water content of the nanoemulsion may vary according to the desired properties. In some embodiments, the water content is about 10% to about 90% by weight, based on the total weight of the nanoemulsion. In some embodiments, the water content is from about 15% to about 60% by weight, such as from about 20% to about 50% by weight, about 25% to about 50% by weight, or about 30% to about 50% by weight, based on the total weight of the nanoemulsion.

In some embodiments, one or more hydrophilic, water soluble components may be added to the water, including short chain mono-, di-, and polyhydric alcohols, (e.g., ethanol, benzyl alcohol, glycerol, propylene glycol, propylene carbonate, polyethylene glycol with an average molecular weight of about 200 to about 10,000, diethylene glycol monoethyl ether, and combinations thereof). While in some nanoemulsions provided herein can include mono-alcohols (e.g., ethanol), the nanoemulsions can, in some embodiments, be formulated with substantially no mono-alcohol (e.g., no ethanol) or no mono-alcohol (e.g., no ethanol). By “substantially no mono-alcohol” is meant that no mono-alcohol (e.g., no ethanol) is intentionally added to the nanoemulsion. For example, certain embodiments can be characterized as having less than 0.001% by weight of mono-alcohols, or less than 0.0001%, or even 0% by weight of mono-alcohols.

### Additional, Optional Components

In addition to the components referenced herein above (i.e., an active ingredient, a surfactant, an oil component, and water), the nanoemulsions provided herein can comprise any number of additional optional components. Such additional optional components include, but are not limited to, humectants, antioxidants, sweeteners, taste modifying agents, flavorants, colorants, salts, and combinations thereof.

A humectant can be added, e.g., to reduce the overall water activity of the oral product, and thus further improve the stability and shelf-life of the product (e.g., by helping to prevent microbial growth within the nanoemulsion). Examples of humectants include, but are not limited to, polyols (e.g., glycerin, 1,2-propanediol (propylene glycol), 1,3-propanediol, dipropylene glycol, sorbitol, xylitol, mannitol, and mixtures thereof). Particularly advantageous polyols are those that are miscible in water. In some embodiments, the nanoemulsion comprises a humectant selected from the group consisting of glycerin, propylene glycol, and mixtures thereof. Advantageously, in some embodiments, the humectant (e.g., glycerin) can also function as a preservative within the disclosed nanoemulsions, endowing them with microbial stability. The humectant (such as glycerin and/or propylene glycol) may be present in an amount of 0% to about 50% by weight of the nanoemulsion, such as about 1% to about 40% by weight of the nanoemulsion, such as from about 10% to about 40% by weight, from about 20% to about 40% by weight of the oral product, or about 20% to about 30% by weight of the nanoemulsion.

Antioxidants are particularly useful in some embodiments to stabilize an active ingredient (e.g., a cannabinoid or cannabimimetic) within the disclosed nanoemulsions. As used herein, the term “antioxidant” refers to a substance which prevents or suppresses oxidation by terminating free radical reactions, and may delay or prevent some types of cellular damage. Antioxidants may be naturally occurring or synthetic and can be lipophilic or non-lipophilic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, Gingko biloba, Saint John's Wort, saw palmetto, green tea, black tea, black cohosh, cayenne, chamomile, cloves, cocoa powder, cranberry, dandelion, grapefruit, honeybush, Echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, Papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, Yerba mate, yerba santa, Bacopa monniera, Withania somnifera, Lion's mane, and Silybum marianum. Such botanical materials may be provided in fresh or dry form, essential oils, or may be in the form of an extract. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, ascorbyl esters (e.g., ascorbyl palmitate or ascorbyl stearate), sodium ascorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the nanoemulsion.

Sweeteners can optionally be incorporated within the disclosed nanoemulsions in natural or artificial form or as a combination of artificial and natural sweeteners. Examples of natural sweeteners include fructose, sucrose, glucose, maltose, dextrose, fructose, mannose, galactose, lactose, Stevia, honey, and the like. Examples of artificial sweeteners include sucralose, isomaltulose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, Stevia is particularly preferable as a sweetener within the disclosed nanoemulsions. Although not intending to be limited by theory, it is believed that Stevia, due to its lipophilic structure, has a higher likelihood of being solubilized/present within the dispersed phase (along with the active ingredient); as such, especially with bitter active ingredients, it may provide particularly beneficial sweetening properties. When present, a sweetener or combination of sweeteners may make up from about 0.1% to about 5% of the nanoemulsion, e.g., about 0.1% to about 2% of the nanoemulsion, or about 0.1% to about 1% of the nanoemulsion by weight.

Taste modifying agents (also referred to as “taste modifiers”) can optionally be included, e.g., to mask the bitterness of one or more components of the nanoemulsion. For example, in some embodiments, inclusion of a taste modifier can be useful to mask the bitterness of a cannabinoid or cannabimimetic in the nanoemulsion. The taste modifying agent may improve the organoleptic properties of a nanoemulsion as disclosed herein, and may serve to mask, alter, block, or improve e.g., the flavor of a nanoemulsion as described herein. Non-limiting examples of such taste modifiers include analgesic or anesthetic herbs, spices, and flavors which produce a perceived cooling (e.g., menthol, Eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the cannabinoid has a bitter taste, and the oral product comprises a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., a cannabinoid or cannabimimetic). In some embodiments, the taste modifier is capsaicin. In some embodiments, the taste modifier is the amino acid gamma-amino butyric acid (GABA), referenced herein above with respect to amino acids. Studies in mice suggest that GABA may serve function(s) in taste buds in addition to synaptic inhibition. See, e.g., Dvoryanchikov et al., J. Neurosci. 2011 Apr. 13; 31(15):5782-91. Without wishing to be bound by theory, GABA may suppress the perception of certain tastes, such as bitterness. In some embodiments, the taste modifier is adenosine monophosphate (AMP). AMP is a naturally occurring nucleotide substance which can block bitter food flavors or enhance sweetness. It does not directly alter the bitter flavor, but may alter human perception of “bitter” by blocking the associated receptor. In some embodiments, the taste modifier is lactisole. Lactisole is an antagonist of sweet taste receptors. Temporarily blocking sweetness receptors may accentuate e.g., savory notes. One particularly useful taste modifier to affect the perception of a bitter taste associated with certain cannabinoids (e.g., CBD) is a glycyrrhizinate salt, e.g., monoammonium glycyrrhizinate (MAG), e.g., sold under the tradename MAGNASWEET®. In some embodiments, MAG can further ameliorate aftertaste associated with certain sweeteners, e.g., Stevia, where incorporated within the nanoemulsion. Suitable MAG products include, but are not limited to, Magnasweet CM2 and Magnasweet CM1, as well as Magnasweet MM100, MM100F, MM110, MM110F, MM-100NF, MM100-EP, MM200F, and MM210F; selection may depend, at least in part, on the flavor(s) to be modified within the nanoemulsion.

When present, a representative amount of taste modifier is about 0.01% by weight or more, about 0.05% by weight or more, or about 0.1% by weight or more, but will typically make up less than about 5% by weight of the total weight of the nanoemulsion (e.g., from about 0.01% to about 2%, about 0.05% to about 1%, or about 0.1% to about 0.5% by weight of the total weight of the nanoemulsion).

Optionally, flavorants can be included within the disclosed nanoemulsions. Flavorants can also be referred to as “flavor materials,” “flavors,” “flavorings,” or “flavoring agents”). A wide range of flavorants are known. Flavorants are any flavorful or aromatic substances that are capable of altering the sensory characteristics associated with the nanoemulsions. Examples of sensory characteristics that can be modified by the flavorants include, taste, mouthfeel, moistness, coolness/heat, and/or fragrance/aroma.

Flavorants can be natural or synthetic, and the character of these flavors can be described as, without limitation, fresh, sweet, herbal, confectionary, floral, fruity, spice, spicy. Such flavoring agents can, in some embodiments, be employed as concentrates or flavor packages. Some examples of flavorants include, but are not limited to, vanilla, vanillin, ethyl vanillin, cream, tea, coffee, fruit (e.g., apple, cherry, strawberry, peach and citrus flavors, including lime, lemon, and orange), maple, menthol, mint, peppermint, spearmint, wintergreen, nutmeg, clove, lavender, cardamom, ginger, honey, anise, sage, rosemary, hibiscus, rose hip, Yerba mate, guayusa, honeybush, rooibos, yerba santa, Bacopa monniera, Gingko biloba, Withania somnifera, cinnamon, Eucalyptus, sandalwood, jasmine, cascarilla, coffee, cocoa/chocolate, licorice, and flavorings and flavor packages of the type and character traditionally used for the flavoring of cigarette, cigar, and pipe tobaccos. Some examples of plant-derived compositions that may be suitable are disclosed in U.S. Pat. No. 9,107,453 and U.S. Pat. App. Pub. No. 2012/0152265 both to Dube et al., the disclosures of which are incorporated herein by reference in their entireties. The selection of such flavoring components is variable based upon factors such as the sensory characteristics that are desired for the nanoemulsion, their solubility, and other physiochemical properties. The present disclosure is intended to encompass any such further components that are readily apparent to those skilled in the art of tobacco and tobacco-related or tobacco-derived products. See, e.g., Gutcho, Tobacco Flavoring Substances and Methods, Noyes Data Corp. (1972) and Leffingwell et al., Tobacco Flavoring for Smoking Products (1972), the disclosures of which are incorporated herein by reference in their entireties. It should be noted that reference to a flavorant should not be limited to any single flavorant as described above, and may, in fact, represent a combination of one or more flavorants. Additional flavorants, flavoring agents, additives, and other possible enhancing constituents are described in U.S. Pat. App. Pub. No. 2019/0082735 to Phillips et al., which is incorporated herein by reference in its entirety.

In some embodiments, flavorants are plant extracts. Extracts selected for use in certain embodiments of the disclosed methods and materials can be derived from a variety of species, using a variety of techniques that produce extract in a variety of usable forms, such as a tobacco extract or similar flavor being derived from a plant of the Nicotiana species. As used herein, the term “tobacco extract” means components separated from, removed from, or derived from, tobacco using tobacco extraction processing conditions and techniques. Purified extracts of tobacco or other botanicals specifically can be used. Typically, tobacco extracts are obtained using solvents, such as solvents having an aqueous nature (e.g., water) or organic solvents (e.g., alcohols, such as ethanol or alkanes, such as hexane). As such, extracted tobacco components are removed from tobacco and separated from the unextracted tobacco components; and for extracted tobacco components that are present within a solvent, (i) the solvent can be removed from the extracted tobacco components, or (ii) the mixture of extracted tobacco components and solvent can be used as such. Examples of types of tobacco extracts, tobacco essences, solvents, tobacco extraction processing conditions and techniques, and tobacco extract collection and isolation procedures, are set forth in Australia Pat. No. 276,250 to Schachner; U.S. Pat. No. 2,805,669 to Meriro; U.S. Pat. No. 3,316,919 to Green et al.; U.S. Pat. No. 3,398,754 to Tughan; U.S. Pat. No. 3,424,171 to Rooker; U.S. Pat. No. 3,476,118 to Luttich; U.S. Pat. No. 4,150,677 to Osborne; U.S. Pat. No. 4,131,117 to Kite; U.S. Pat. No. 4,506,682 to Muller; U.S. Pat. No. 4,986,286 to Roberts et al.; U.S. Pat. No. 5,005,593 to Fagg; U.S. Pat. No. 5,065,775 to Fagg; U.S. Pat. No. 5,060,669 to White et al.; U.S. Pat. No. 5,074,319 to White et al.; U.S. Pat. No. 5,099,862 to White et al.; U.S. Pat. No. 5,121,757 to White et al.; U.S. Pat. No. 5,131,415 to Munoz et al.; U.S. Pat. No. 5,230,354 to Smith et al.; U.S. Pat. No. 5,235,992 to Sensabaugh; U.S. Pat. No. 5,243,999 to Smith; U.S. Pat. No. 5,301,694 to Raymond; U.S. Pat. No. 5,318,050 to Gonzalez-Parra et al.; U.S. Pat. No. 5,435,325 to Clapp et al.; and U.S. Pat. No. 5,445,169 to Brinkley et al., which are incorporated herein by reference in their entireties.

In some embodiments, flavor components that can be incorporated within a nanoemulsion as provided herein comprise one or more alcohols, aldehydes, aromatic hydrocarbons, ketones, esters, terpenes, terpenoids, trigeminal sensates. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol.

In some embodiments, the flavorant comprises menthol, spearmint and/or peppermint. In some embodiments, the flavorant comprises flavor components of cucumber, blueberry, citrus fruits and/or redberry. In some embodiments, the flavorant comprises eugenol. In some embodiments, the flavorant comprises flavor components extracted from tobacco. In some embodiments, the flavorant comprises flavor components extracted from Cannabis.

In some embodiments, the flavorant may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to, eucolyptol or WS-3. Flavorants, including extracts, may be provided in various forms, e.g., a liquid form or a substantially solid (e.g., powder or pellet-type) form.

The quantity of flavorant present within the nanoemulsions of the present disclosure may vary. When the nanoemulsions comprise one or more flavorants, the content of such flavorants is generally up to about 10% by weight of the nanoemulsion, e.g., up to about 5% by weight, up to about 2% by weight, or up to about 1% by weight. For example, a flavorant may be present in a quantity of from about 0.01%, about 0.05%, about 0.1%, or about 0.5%, to about 2%, about 5%, about 8%, or about 10% by weight of the final nanoemulsion.

A colorant may optionally be employed in amounts sufficient to provide the desired physical attributes to the nanoemulsion. Examples of colorants include various dyes and pigments, such as caramel coloring and titanium dioxide. The amount of colorant utilized in the product can vary, but when present is typically up to about 3% by weight, such as from about 0.01%, about 0.1%, about 0.5%, or about 1%, to about 3% by weight, based on the total weight of the nanoemulsion.

In some embodiments, the nanoemulsions comprise a salt (e.g., an alkali metal salt), typically employed in an amount sufficient to provide desired sensory attributes to the product. In some embodiments, certain salts may also serve as electrolytes or act in synergy with electrolytes. For example, without wishing to be bound by theory, sodium citrate may provide both a source of sodium (electrolyte) as well as aid in the absorption of other electrolytes and water. Non-limiting examples of suitable salts include sodium chloride, potassium chloride, ammonium chloride, flour salt, sodium acetate, sodium citrate, and the like. In some embodiments, the salt is sodium chloride, ammonium chloride, sodium citrate, or a combination thereof. In some embodiments, the salt is sodium chloride.

When present, a representative amount of salt is about 0.1% by weight or more, about 0.5% by weight or more, about 1.0% by weight or more, or about 1.5% by weight or more, but will typically make up about 10% or less of the total weight of the nanoemulsion, or about 7.5% or less, or about 5% or less (e.g., from about 0.5 to about 5% by weight). In specific embodiments, the product comprises sodium chloride in an amount by weight of from about 1 to about 3%, based on the total weight of the nanoemulsion.

The disclosed nanoemulsions can be formed in various manners. Generally, nanoemulsions can be provided by combining the ingredients thereof and mixing/emulsifying. The ingredients can be mixed in various orders, and the mixing/emulsifying can be promoted in various ways.

In one embodiment, the disclosed nanoemulsions are prepared by combining the oil-soluble components and the aqueous-soluble components separately. The surfactants and second oil (e.g., lecithin) are then added to the aqueous mixture, which is then subjected to mixing using a high shear mixer. The oil-soluble component mixture is slowly added to the aqueous/surfactant mixture under the same high shear mixing condition. The full mixture is then subjected to energy to decrease the droplet size (e.g., via sonication).

Both high-energy and low-energy emulsification methods are known for the production of nanoemulsions. Suitable, non-limiting high-energy emulsification methods include, but are not limited to, high-energy stirring, ultrasonic emulsification, high-pressure homogenization, microfluidization, and membrane emulsification. Suitable non-limiting low-energy emulsification methods include, but are not limited to, phase inversion temperature, emulsion inversion point, and spontaneous emulsification.

The nanoemulsions described herein advantageously exhibit high stability. High physical stability and shelf life can be evidenced, e.g., by the maintenance of a similar particle (droplet/micelle) size within the nanoemulsion over time. Unstable formulations tend to grow in micelle size over time and eventually phase separate over time. As such, high physical stability and shelf life can be evaluated, e.g., by comparing particle size over time and/or by physical observation (i.e., looking to determine whether any significant phase separation, e.g., via flocculation and/or creaming of the dispersed phase, has occurred within the nanoemulsion).

In some embodiments, an accelerated aging study can be used as indicative of good shelf life and high physical stability. For example, in some embodiments, the nanoemulsions described herein can be subjected to an accelerated aging study (e.g., as set forth in ASTM D1791-93) with little change in droplet (dispersed phase) size. For example, in some embodiments, the disclosed nanoemulsions exhibit a droplet Z-average diameter of about 90 nm or less or about 100 nm or less, even after aging.

In some embodiments, the shelf life of the nanoemulsion may be at least about 6 months, at least about 7 months, at least about 8 months, at least about 9 months, at least about 10 months, at least about 11 months, or at least about 12 months. As described herein, the “shelf-life” refers to the period of time during which no visible microbiological growth is observed on the product, and there is no deterioration in the appearance and/or taste of the nanoemulsion (e.g., as indicated by observable creaming/phase separation and/or by significant growth of the droplet particle size of the nanoemulsion).

### Tinctures

The disclosed nanoemulsions can be used directly (i.e., with no further modification) as a tincture of the active ingredient. When used directly, the tincture can be used in varying amounts and at various number of times per day. In some embodiments, a subject (human or animal) may take 2-5 mL of the tincture, 1-5 times per day. Most typically, the tincture is used orally. In some embodiments, the tincture is provided within a bottle equipped with a dropper; the subject may apply the tincture via the dropper into the mouth, such as on the tongue or cheek (such that the active ingredient is absorbed through the mucosa/buccally), swallowed (such that the active ingredient is taken up by intestinal absorption/enterally), or applied under the tongue (such that the active ingredient is absorbed through the mucosa/sublingually). In some embodiments, the subject adds the tincture to a food or beverage, e.g., at the point of service.

### Other Oral Products

In some embodiments, the disclosed nanoemulsions can be further formulated as other oral products, or for inclusion within other oral products. For example, the nanoemulsions can be formulated as drinkable shots, or for inclusion within, e.g., a liquid-filled capsule.

Oral products in the form of shots are provided, e.g., by diluting a nanoemulsion as provided herein above. Shots can be provided/packaged for consumption as a single shot (e.g., about 25 mL to about 75 mL) or in greater volume that can be used as multiple shots. Typically, no particular processing methods are required; simple mixing is, in some embodiments, sufficient to provide a shot. The amount of dilution can vary; in some embodiments, about 0.5% to about 5% of the shot by weight comprises a nanoemulsion as described herein above.

The composition of the remainder of the shot can vary. In some embodiments, the remaining components comprise or consist essentially of water, as well as one or more preservatives, one or more pH adjusters/buffering agents (which can function, e.g., to inhibit microbial growth within the liquid), and/or one or more antioxidants. In some embodiments, shots can further comprise one or more sweeteners as referenced above (e.g., in an amount of up to about 2% by weight) and/or one or more flavors (e.g., in an amount of up to about 3% by weight). In some embodiments, the shots can further comprise one or more terpenes. Although in some embodiments, such shots are considered non-alcoholic, in other embodiments, an amount of mono-alcohol can optionally be included within the disclosed shots (replacing a portion or all of the water). In some embodiments, the shot is carbonated, which may further enhance stability of the product by removing oxygen. Further flavorants, colorants, sweeteners, and the like can, in some embodiments, be included within the shots as provided herein.

Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Non-limiting examples of suitable buffers include alkali metals acetates, glycinates, phosphates, glycerophosphates, citrates, carbonates, hydrogen carbonates, borates, or mixtures thereof. In certain embodiments, the buffering agent comprises a sodium citrate/citric acid buffer system. The buffering agent is typically present in an amount less than about 5% by weight, based on the weight of the shot, for example, from about 0.1% to about 5%, such as, e.g., from about 0.01% to about 1% by weight, from about 0.01% to about 0.5% by weight, or from about 0.01% to about 0.1% by weight, based on the total weight of the shot.

Examples of preservatives include, but are not limited to, potassium sorbate, sodium benzoate, calcium propionate, combinations thereof, and the like. In some embodiments, preservatives include, e.g., benzyl alcohol, cetylpyridine chloride, glycerin, methyl paraben, propylene glycol, propylene paraben, potassium sorbate, sodium benzoate, sorbic acid, sodium propionate, or combinations thereof. In some embodiments, such preservatives can help to decrease the water activity of the shot, further improving the stability and shelf life of the shot. In particular embodiments, the disclosed shots comprise sodium benzoate and potassium sorbate as preservatives. The preservative (or preservatives) is typically present in an amount less than about 5% by weight, based on the weight of the shot, for example, from about 0.1% to about 5%, such as, e.g., from about 0.01% to about 1% by weight, from about 0.01% to about 0.5% by weight, or about 0.05% to about 0.5% by weight, based on the total weight of the shot.

Examples of antioxidants include, but are not limited to, those referenced above with respect to nanoemulsions. In some embodiments, the shots described herein comprise both a water-soluble antioxidant (e.g., sodium ascorbate) and an oil-soluble antioxidant (e.g., Vitamin E). The antioxidant (or antioxidants) is typically present in an amount less than about 5% by weight, based on the weight of the shot, for example, from about 0.1% to about 5%, such as, e.g., from about 0.01% to about 1% by weight, from about 0.01% to about 0.5% by weight, or about 0.05% to about 0.5% by weight, based on the total weight of the shot.

As with the nanoemulsions/tinctures referenced above, a shot as provided herein can, in some embodiments, exhibit a shelf life of at least about 6 months, at least about 7 months, at least about 8 months, at least about 9 months, at least about 10 months, at least about 11 months, or at least about 12 months.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### Examples

Aspects of the present invention are more fully illustrated by the following examples, which are set forth to illustrate certain aspects of the present invention and are not to be construed as limiting thereof.

### Example 1: Nanoemulsion Preparation

A nanoemulsion comprising the components listed in Table 1 was prepared by the following method. The oil-soluble components are combined and the aqueous-soluble components are separately combined. Poly oxyl 40 stearate and canola lecithin are then added to the aqueous mixture, which is then subjected to mixing using a high shear mixer. The oil-soluble component mixture is slowly added to the aqueous/surfactant mixture under the same high shear mixing condition, forming droplets within the continuous aqueous phase with diameters of about 200 nm. The full mixture is then subjected to sonification to reduce the droplet size, giving the desired nanoemulsion.

TABLE 1
Nanoemulsion Composition (CBD)

| Component | Amount (per 100 g batch) | |
|---|---|---|
| Poly oxyl 40 stearate | 8-12 | g |
| Canola lecithin | 10-15 | g |
| Sunflower oil | 5-10 | g |
| CBD isolate | 2-4 | g |
| Glycerin | 20-40 | g |
| Stevia | 0.2-2 | g |
| MagnaSweet | 0.1-1 | g |
| Tocopherol | 0.2-2 | g |

| | |
|---|---|
| Deionized Water | Remainder |

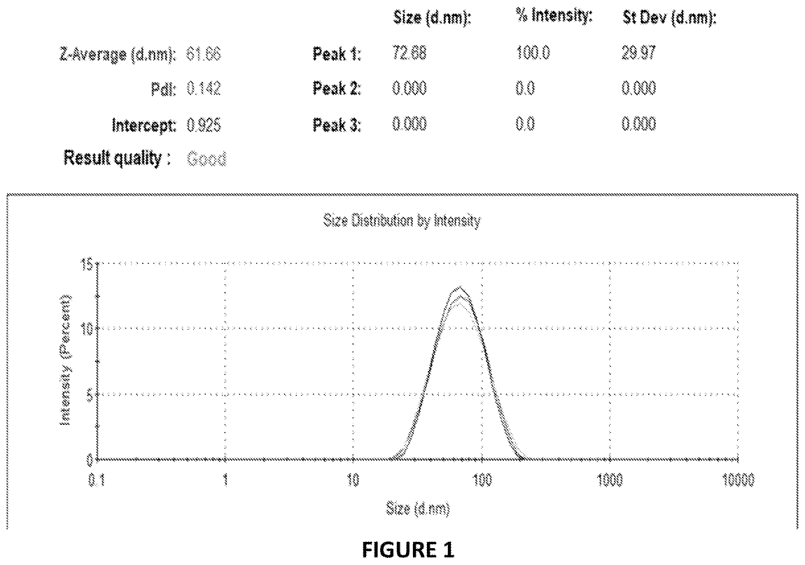

The nanoemulsion was evaluated by dynamic light scattering to determine the average micelle size (diameter) within the liquid, as shown, e.g., in FIG. 1. The evaluation demonstrated that the prepared nanoemulsion had a Z-average particle size of 61.66 nm, indicating high bioavailability.

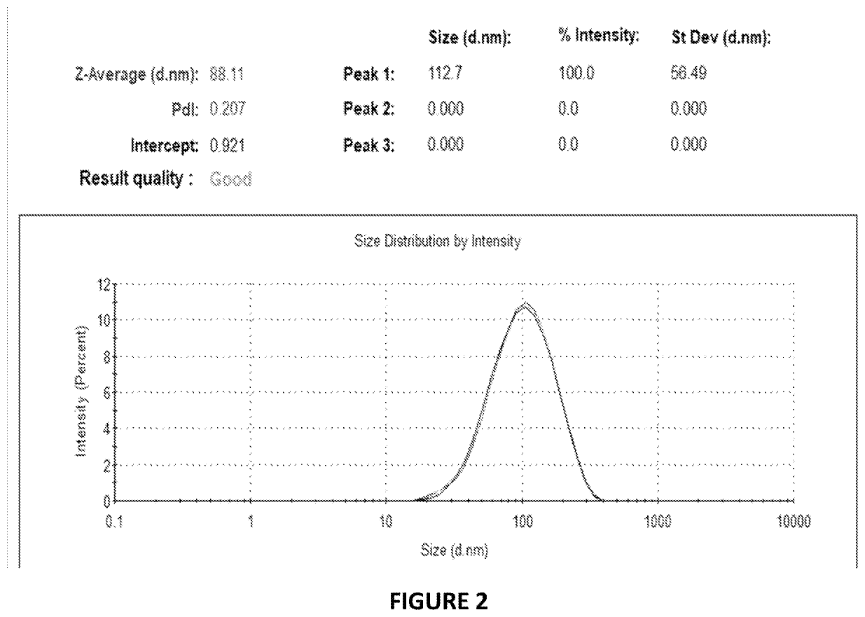

The nanoemulsion was then subjected to accelerated aging studies (via subjecting the nanoemulsion to elevated temperatures or gravitational forces/centrifugation) over a predetermined time period, and monitoring the micelle size over time. This study uses the physical stability of an emulsion at high temperature to predict the emulsion's stability at room temperature over time. The nanoemulsion was aged and tested based on ASTM D1791-93, an industry standard for accelerated aging studies). The standard concludes that if an oil-in-water emulsion is physically stable at 52° C. for 30 days, it is predicted to have a stability of 1 year at room temperature. As shown in FIG. 2, after being subjected to the aging conditions associated with ASTM D1791-93, the prepared nanoemulsion had a Z-average micelle size of 88.1 nm, indicating physical stability and bioavailability for a product shelf life of one year.

The nanoemulsion was also monitored by physical observation over time. The formulation of Table 1, above, did not phase separate with time, e.g., over a period of at least three months. By contrast, nanoemulsions prepared with sunflower lecithin powder, soy lecithin powder, or purified phosphatidylcholine in place of the (crude, unrefined) canola lecithin began to show phase separation over time (indicated by creaming of the nanoemulsion at the top of the vials in which the nanoemulsions were contained).

### Example 2: Shot Preparation

A shot comprising the components listed in Table 2 was prepared by simple mixing. The nanoemulsion easily went into water by mixing, and the additional components were added (i.e., preservatives and the buffer system to modify the pH). In some embodiments, one or more antioxidants are also added (e.g., Vitamin E, which is soluble in the oil phase and sodium ascorbate, which is soluble in the water phase).

TABLE 2
Shot Composition (CBD)

| Component | Amount (per 60 mL shot) | |
|---|---|---|
| Nanoemulsion (see Table 1) | 1-2 | g |
| Sodium benzoate | 0.01-1 | g |
| Potassium Sorbate | 0.01-1 | g |
| Citric acid | 0.01-1 | g |
| Sodium Citrate | 0.01-1 | g |

| | |
|---|---|
| Deionized Water | Remainder |

The shot was evaluated as for the nanoemulsion above, and was found to have the same micelle size as the nanoemulsion.

A first sample of the shot was subjected to an accelerated aging experiment at 70° C. for 2 weeks. Turbidity measurements of the shot were made using a turbidimeter over the course of the 2 weeks, indicating an average turbidity of 210±20 NTU. Turbidity is an indication of micelle size with time; consistent turbidity (as demonstrated in this study) indicates physical stability of micelles.

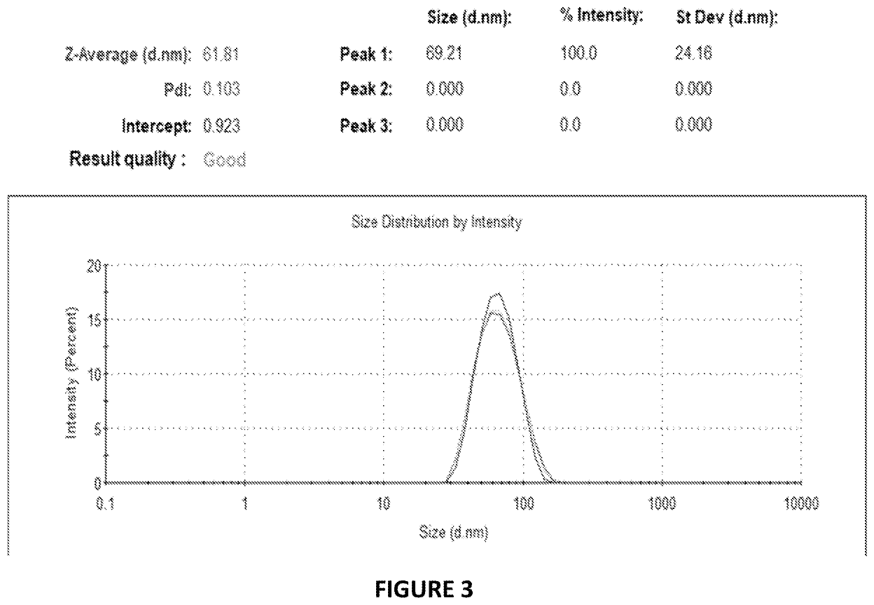

A second sample of the shot was subjected to the aging conditions of ASTM D1791-93 (52° C. for 30 days). The prepared shot had a Z-average micelle size of 62 nm after aging, indicating physical stability and bioavailability for a product shelf life of one year, as shown in FIG. 3. A USP 51 Microbial Challenge test was used to determine whether the shot formulation preservatives were robust enough to protect from microbial contaminants. The shot formula uses the preservatives sodium benzoate and potassium sorbate in a sodium citrate/citric acid buffer system to inhibit microbial growth. The USP 51 microbial challenge inoculates the microbials Pseudomonas aeruginosa, E. coli, Staphylococcus aureus, Candida albicans and Aspergillus brasiliensis within the CBD shot at Day 0. Over a period of 28 days, the microbial count of the shot is measured. The results are provided in Table 3, below, and demonstrate that the preservatives were extremely successful at killing and inhibiting the growth of the microbes within the shot.

TABLE 3
Microbial Count of Inoculated Shot over a 28-Day Period

| | Day 0 | Day 14 | Day 28 |
|---|---|---|---|
| | (CFU/mL) | (CFU/mL) | (CFU/mL) |
| Shot w/Preservative | 3.6E+06 | 150 | 490 |
| Shot w/o Preservative | 3.6E+06 | 3.00E+17 | 2.10E+18 |

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method steps or system elements, it should be understood that those steps and those elements may be combined in other ways to accomplish the same objectives.

Furthermore, those skilled in the art should appreciate that the parameters and configurations described herein are examples only and that actual parameters and/or configurations will depend on the specific application in which the systems and techniques of the invention are used. Those skilled in the art should also recognize or be able to ascertain, using no more than routine experimentation, equivalents to the specific embodiments of the invention. It is, therefore, to be understood that the embodiments described herein are presented by way of example only and that, within the scope of any appended claims and equivalents thereto; the invention may be practiced other than as specifically described.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements.

1. A nanoemulsion, comprising:
a cannabinoid and/or a cannabimimetic;
a first oil;
a second oil; and
water,
wherein the cannabinoid or cannabimimetic is present in an amount of about 1% by weight or greater.

2. The nanoemulsion of claim 1, wherein the cannabinoid or cannabimimetic is present in an amount of about 2% by weight or greater, e.g., about 2% to about 3% by weight.

3. The nanoemulsion of claim 1, wherein the cannabinoid or cannabimimetic is present in an amount of about 1% to about 4% by weight.

4. The nanoemulsion of claim 1, wherein the cannabinoid or cannabimimetic is selected from the group consisting of cannabigerols, cannabichromenes, cannabidiols, tetrahydrocannabinols, cannabinols, cannabinodiols, and combinations thereof.

5. The nanoemulsion of claim 1, wherein the cannabinoid or cannabimimetic is selected from the group consisting of cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabmolic acid (THCA), tetrahydrocannabivarinic acid (THCV A), and combinations thereof.

6. The nanoemulsion of claim 1, wherein the cannabinoid or cannabimimetic comprises cannabidiol (CBD).

7. The nanoemulsion of claim 1, wherein the cannabinoid or cannabimimetic is selected from the group consisting of yangonin, alpha-amyrin or beta-amyrin (also classified as terpenes), cyanidin, curcumin (turmeric), catechin, quercetin, salvinorin A, N-acylethanolamines, N-alkylamide lipids, and combinations thereof.

8. The nanoemulsion of claim 1, wherein first oil comprises sunflower oil.

9. The nanoemulsion of claim 1, wherein the second oil comprises lecithin.

10. The nanoemulsion of claim 9, wherein the second oil is canola lecithin.

11. The nanoemulsion of claim 1, wherein the weight ratio of the second oil to the first oil is about 1.5 or greater (e.g., about 1.5 to about 3).

12. The nanoemulsion of claim 1, further comprising a surfactant.

13. The nanoemulsion of claim 12, wherein the surfactant is a polyoxyethylene stearate.

14. The nanoemulsion of claim 1, further comprising one or more natural or artificial sweeteners selected from the group consisting of saccharin, acesulfame K, aspartame, sucralose, isomalt, lactose, mannitol, sorbitol, xylitol, sucrose, Stevia, and combinations thereof.

15. The nanoemulsion of claim 14, wherein the natural or artificial sweetener comprises Stevia.

16. The nanoemulsion of claim 1, further comprising a bitterness suppressant.

17. The nanoemulsion of claim 16, wherein the bitterness suppressant is a glycyrrhizic acid or a salt thereof.

18. The nanoemulsion of claim 1, further comprising an antioxidant selected from the group consisting of citric acid, Vitamin E, a tocopherol, epicatechol, epigallocatechol, epigallocatechol gallate, erythorbic acid, sodium erythorbate, ascorbyl palmitate, ascorbyl stearate, sodium ascorbate, ascorbic acid, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof

19. The nanoemulsion of claim 1, further comprising a humectant.

20. The nanoemulsion of claim 19, wherein the humectant is a polyol.

21. The nanoemulsion of claim 19, wherein the humectant comprises glycerin, propylene glycol, 1,3-propanediol, dipropylene glycol, sorbitol, xylitol, mannitol, or a combination thereof.

22. The nanoemulsion of claim 1, wherein the nanoemulsion is substantially free of a mono-alcohol.

23. The nanoemulsion of claim 1, wherein the nanoemulsion comprises a dispersed phase within the water, wherein the dispersed phase has an average droplet size of about 100 nm or less.

24. The nanoemulsion of claim 1, wherein the nanoemulsion exhibits physical stability for 6 months or more or for 12 months or more.

25. An oral product in the form of a tincture, consisting essentially of the nanoemulsion of claim 1.

26. An oral product in the form of a shot, comprising the nanoemulsion of claim 1, diluted in water.

27. The oral product of claim 26, further comprising at least one buffering agent, at least one preservative, at least one antioxidant, or any combination thereof.

28. The oral product of claim 26, wherein the shot exhibits physical stability for 6 months or more or for 12 months or more.
